(19) United States
(12) Patent Application Publication
(10) Pub. No.: US 2018/0200103 A1
(43) Pub. Date: Jul. 19, 2018

(54) FULL BREATH NO SNORE

(71) Applicant: Silent Sleep Group, LLC, San Antonio, TX (US)

(72) Inventor: Bryan Keropian, Encino, CA (US)

(21) Appl. No.: 15/871,319

(22) Filed: Jan. 15, 2018

Related U.S. Application Data
(60) Provisional application No. 62/447,594, filed on Jan. 18, 2017.

(51) Int. Cl. A61F 5/56; A61B 13/00

(52) U.S. Cl. CPC A61F 5/566; A61B 13/00

(57) ABSTRACT

A dental oral appliance to open the airway for a sleeping individual who suffers with snoring, comprising affixing an appliance to the upper or lower teeth, a bar that extends from the inside of the right side teeth and the left side teeth, a bendable wire tail affixed to the bar, extending back from the bar, with a plastic plate affixed to the posterior end of the wire tail to depress the tongue.

### CROSS-REFERENCE TO RELATED APPLICATIONS

This application claims the benefit of U.S. Provisional Application No. 62/447,594, filed Jan. 18, 2017, the disclosure of which is incorporated herein by reference in its entirety.

### TECHNICAL FIELD

This invention is an over the counter snore appliance that is adjustable by the wearer to reduce or eliminate snoring without advancing the mandible.

### BACKGROUND

It has been estimated that ninety million American adults and children snore and that three in every ten adults snore. Snoring can have serious medical consequences for some people. Snoring is the first indication of a potentially life-threatening sleep disorder called Obstructive Sleep Apnea. If not diagnosed or if left untreated, Obstructive Sleep Apnea could result in severe medical consequences such as systemic high blood pressure, cardiovascular disease and even sudden death. The Applicant herein has several patents relating to dental oral appliances to open the airway for a sleeping individual who suffers with snoring or obstructive sleep apnea, such as U.S. Pat. No. 8,132,567.

### SUMMARY

The dental oral appliance of this invention is a new appliance that provides a new advance in opening the airway for those that suffer from snoring. This single arched appliance fits over a person's upper or lower jaw fitting on one jaw. The back of the appliance has a bar extending across the back of the appliance, running from the inner right side of the person's upper teeth to the inside of the inner left inside of the upper left side teeth. Also, the back of the appliance can have a bar extending across the back of the appliance, running from the inner right side of the person's lower teeth to the inside of the inner left inside of the lower left side teeth. In the middle of the bar is a wire tail inserted into the bar that extends back from the appliance, from ¼ inch to 2 inches. The back of the wire extending out from the bar has a plastic plate. The extended wire can be bent down by the wearer, enabling the plastic plate to depress the tongue (see FIGS. 5, 6). This results in elimination of snoring in 90% of cases.

### BRIEF DESCRIPTION OF THE DRAWINGS

FIG. 1 shows an appliance having a posterior bar with a wire tail and a plastic plate to depress the tongue.

FIG. 2 shows the appliance with a boil and bite material in the body of the appliance.

FIG. 3 shows the inside of the body of the appliance, the shell, after the user has bitten down on the boil and bite material, where individual indentations for each tooth can be seen.

FIG. 4 shows the metal tail behind the appliance showing that the metal tail is very strong, straight back with no bend.

FIG. 5 shows that the metal tail is very strong and also flexible and can be bent by the user, down for the comfort and effect position to eliminate snoring.

FIG. 6 shows that the tail can be bent downward extensively, dependent on the needs and comfort of the user.

### DETAILED DESCRIPTION OF THE INVENTION

All of the common over the counter snore appliances advance the mandible and force the lower jaw forward to open the airway to stop snoring. This can cause pain and can cause irregularities with the correct meshing of the teeth. With the sleep appliance of this invention this does not happen. The patient wears the appliance in their normal bite, depresses the tongue, and has no sleep appliance pain or bite problems. As all of the other over the counter snore appliances work by forcing the mandible forward, with the forward movement there can be pain and changes in the bite that could require extensive dental work or orthodontics. With this described snore appliance the patient has a normal bite and no debilitating changes occur.

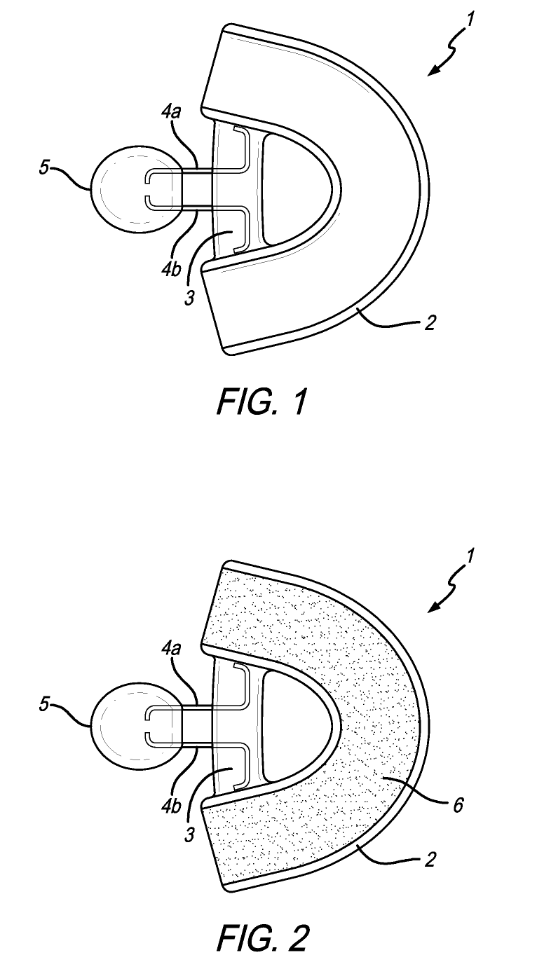

Referring to the drawings in FIG. 1, there is shown an appliance 1 comprising the empty shelled body 2 of the appliance that fits over the upper teeth or lower teeth. A transpalatal, or translingual, posterior plastic bar 3 extends across the back of the appliance 1, running from the inner right side of the person's upper teeth to the inner left inside of the upper left side teeth. Plastic bar 3 can also run from the inner right side and inner left side of the person's lower teeth. The transpalatal, or translingual, bar 3 holds a two wire tail 4a, 4b, having a plastic plate, 5 at the end of the wire tail 4a, 4b. The wire tail 4a, 4b and the plastic plate 5 act to depress the tongue.

The two wire tail 4a, 4b that extends out from bar 3 has two L shaped wires 4a and 4b, placed to be held and imbedded in posterior bar 3. This L shape prevents rotation of the two wires 4a, 4b in the tail, imbedded in the plastic of posterior bar 3. With this L shape, the wires 4a and 4b cannot be directly pulled out or rotated out of place. They are locked into place by the L shape of the two wires 4a, 4b.

This appliance fits over the upper teeth, or lower teeth. The appliance has a hollow shell 2 of plastic covering the teeth from the posterior teeth on either side of the upper arch, or the lower arch. It is a horseshoe shape of plastic that is fitted to cover all the teeth on the upper arch, or the lower arch (See FIG. 2). The hollow shell 2 has the bar 3, the wire tail 4a, 4b and the plastic plate 5 attached to the shell 2. The plastic plate 5 can have a variety of shapes as determined by the clinical success of each shape.

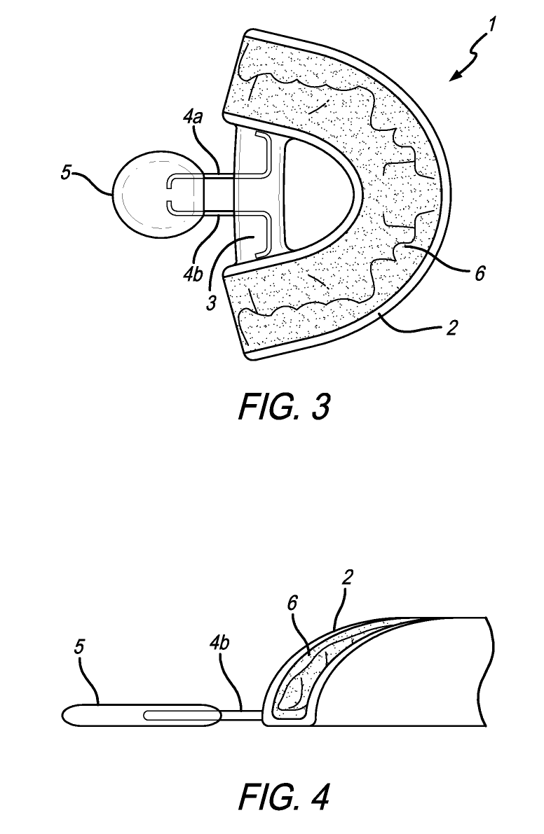

The interior of the hollow plastic shell 2 fitting over the teeth is filled with a boil and bite material. It is a hard material and the entire mouthpiece is filled with the boil bite material and placed in boiling water for about 1 minute. There are numerous boil and bite materials on the market that can be utilized. Elvax by Dupont is an example of this material. The material becomes very soft when boiled. It is then run under water to cool it and it is then fit over the teeth and the covering shell 2 is pressed or bitten down to fit over the teeth. As it is bitten down, the bite is balanced so there is biting on both sides. The bite is done against the flat biting surface 2 of the appliance 1. If the fit isn't correct, it can be redone. The inside shell 2 now has the form of the teeth (See FIG. 3).

The posterior bar 3 on appliance 1 is designed to restrain the tongue and hold the adjustable metal wire tail 4a, 4b and plate 5 in place. The bar 3 can be curved up or down in design to aid in adjusting the wire tail 4a, 4b. The wire tail 4a, 4b can be placed straight back with no bend or it can be bent down by the user for maximum comfort and effective positioning to eliminate snoring. The depression of the tongue is created by the bar 3 or plate 5, or both, of plastic at the posterior end of the wire tail 4a, 4b.

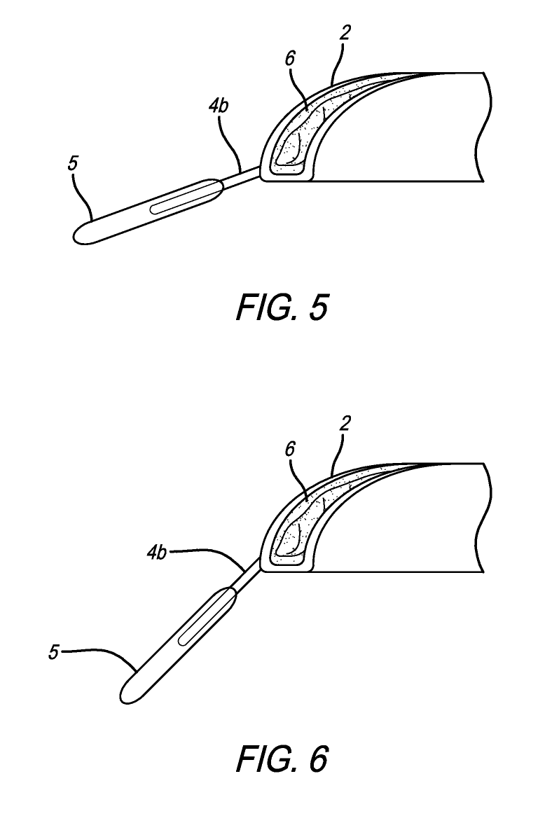

The depressing wire tail 4a, 4b can be from ¼ inch in length to 2 inches in length. It can be placed parallel to the occlusal plane or angled, bent down from the horizontal, any amount down to 5 degrees or any amount down to 90 degrees down toward the tongue. FIG. 4 shows no bend, FIG. 5 shows a 20% bend and FIG. 6 shows a 45% bend. This creates the depression of the tongue to keep the airway open and eliminate snoring. There are numerous orthodontic wires that can be used that have different diameter sizes and strengths. The strongest wires are the best, but may also depend on the patient.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A dental oral appliance to open the airway for a sleeping individual who suffers with snoring, comprising affixing a structure configured to affix the appliance to the upper or lower teeth, a bar that extends from the inside of the right side teeth to the inside of the left side teeth, a bendable wire tail affixed to the bar, extending back from the bar, with a plate of plastic affixed to a posterior end of the wire tail, to depress the tongue and reduce or eliminate snoring.

2. The dental oral appliance of claim 1 in which the bar is a transpalatal bar or a translingual bar.

3. The dental oral appliance of claim 1 in which the structure is a single arched appliance which fits over an individual's upper or lower teeth.

4. The dental oral appliance of claim 3 in which the single arched appliance has a hollow plastic shell able to fit over the teeth, and adapted to be filled with a boil and bite material

5. The dental oral appliance of claim 4 in which the bendable wire tail comprises two L shaped wires embedded into the bar.

6. The dental oral appliance of claim 5 in which the two L shaped wires embedded into the bar can be placed straight back or can be bent down up to 90% as needed.

7. A method for opening the airway for a sleeping individual who suffers with snoring or obstructive sleep apnea, comprising taking a hollow plastic shell, having a bar, a wire tail and a plastic plate, the shell able to being affixed to the upper or lower teeth, filling the entire hollow plastic shell with a boil and bite material and placing the hollow plastic shell in boiling water, then run the shell under water to cool it and then fit the shell over the teeth and pressing down the shell to fit the teeth, and then adjusting the wire tail, and plate to depress the tongue.

8. The method of claim 7 in which the wire tail can be placed straight or adjusted downward.

9. The method of claim 7 in which the bar is a transpalatal bar or a translingual bar.

10. The method of claim 7 in which the bar can be curved up or curved down.

11. The method of claim 8 in which the wire tail can be adjusted downward to as low as 90 degrees.
